(19) United States
(12) Patent Application Publication
(10) Pub. No.: US 2007/0027099 A1
(43) Pub. Date: Feb. 1, 2007

(54) Gene therapy of HBV infection via adeno-associated viral vector mediated long term expression of small hairpin RNA (shRNA)

(76) Inventors: Marie C. Lin, Hong Kong (HK); Ming-Liang He, Hong Kong (HK); Hsiang-Fu Kung, Hong Kong (HK)

Correspondence Address: COOPER & DUNHAM, LLP, 1185 AVENUE OF THE AMERICAS, NEW YORK, NY, 10036, US

(21) Appl. No.: 11/451,876

(22) Filed: Jun. 12, 2006

Related U.S. Application Data
(63) Continuation-in-part of application No. 10/848,768, filed on May 18, 2004.
(60) Provisional application No. 60/471,903, filed on May 19, 2003.

(51) Int. Cl. A61K 48/00

(52) U.S. Cl. 514/44

(57) ABSTRACT

The invention provides a vector comprising an AAV-shRNA vector. The vector is preferably rAAV-151i/1694i. The invention also provides a method of suppressing or inhibiting HBV replication in liver cells infected therewith, comprising administering an amount of an AAVB-shRNA vector effective to suppress, inhibit or reduce HBV replication.

### CROSS-REFERENCE TO RELATED APPLICATION

This is a continuation-in-part of U.S. patent application Ser. No. 10/848,736, filed on May 19, 2004, the entire contents of which are incorporated by reference herein, claiming the benefit of U.S. Provisional Application 60/471,903, filed May 19, 2003.

### FIELD OF THE INVENTION

The invention relates to methods for the delivery of shRNA in vivo by adeno-associated viral vector to treat HBV infections and HBV-associated liver cancer, especially chronic HBV infections.

### BACKGROUND OF THE INVENTION

Hepatitis B virus (HBV) causes estimated 400 million infections worldwide. Chronic HBV infection and HBV-associated hepatocellular carcinoma (HCC) leads to more than one million deaths annually. See Lau G K, “Hepatitis B infection in China,” Clin. Liver Dis. May 2001; 5(2):361-379. Unfortunately, the current treatment of chronic infection is less effective due to low efficacy of the current drugs and occurrence of drug resistance. Therefore, it is urgently needed to develop a novel treatment for HBV infection and HBV-associated liver cancer as new patients are stably increasing.

HBV is a 3.2 kb partially double-stranded, relaxed-circular DNA virus, which encodes polymerase, X protein, core antigen (C), and surface (PreS and S) antigens. All these proteins play important roles in HBV transcriptional regulation, viral packaging, reverse-transcription, and viral recycling; therefore, suppressing these proteins may inhibit HBV reproduction or infectivity. HBV viral genome is highly compact with overlapping open reading frames (ORF), hence targeting one site by RNA interference could inhibit multiple HBV mRNAs expression.

RNAi is a natural process which regulates gene expression via a ubiquitous mechanism. It is initiated by 21- to 23-nucleotide duplex RNA which is homologous in sequence to the target gene and finally leading to the degradation of the target mRNA. In our previous study, we designed short hairpin interfering RNAs (shRNA) to systemically target pregenomic RNA, each individual shRNA targeting either direct repeat (DR) elements, S, core, X, and reverse-transcriptase gene. We showed that HBV replication was significant suppressed. See Chen Y, Du D, Wu J, Chan C P, Tan Y, Kung H F, He ML, “Inhibition of Hepatitis B Virus Replication by Stably Expressed shRNA,” Biochem. Biophys. Res. Commun. Nov. 14, 2003; 311(2):398-404. Other groups showed that synthetic small interfering RNA (siRNA) also potently inhibited HBV replication, although siRNA is very expensive and has a very short half life. See Ying C, De Clercq E, Neyts J., “Selective Inhibition of Hepatitis B Virus Replication by RNA Interference,” Biochem. Biophys. Res. Commun. Sep. 19, 2003; 309(2):482-4; McCaffrey A P, Nakai H, Pandey K, Huang Z, Salazar F H, Xu H, Wieland S F, Marion P L, Kay M A, “Inhibition of Hepatitis B Virus in Mice by RNA Interference,” Nat. Biotechnol. June 2003; 21(6):639-44; Morrissey D V, Lockridge J A, Shaw L, Blanchard K, Jensen K, Breen W, Hartsough K, Machemer L, Radka S, Jadhav V, Vaish N, Zinnen, S, Vargeese C, Bowman K, Shaffer C S, Jeffs L B, Judge A, MacLachlan I, Polisky B, “Potent and Persistent In Vivo Anti-HBV Activity of Chemically Modified siRNAs,” Nat Biotechnol. August 2005; 23(8):1002-7. However, fast viral mutations were generated and viruses could escape the siRNA target if a single siRNA was repeatly administered. See Wu H L, Huang L R, Huang C C, Lai H L, Liu C J, Huang Y T, Hsu Y W, Lu C Y, Chen D S, Chen P J, “RNA Interference-Mediated Control of Hepatitis B Virus and Emergence of Resistant Mutant,” Gastroenterology, March 2005; 128(3):708-16. Therefore, development of effective RNAi delivery system, which could spontaneously deliver several shRNAs targeting several different RNAs, is urgently needed.

Adeno-associated virus (AAV) is one of most promising vectors for gene therapy. The recombinant AAV (rAAV) provides a non-pathogenic and latent infection by integration into the host genome; it also shows high transduction efficiency of both dividing and non-dividing cells and tissues with persistent transgenes expression. See Rabinowitz J E, Samulski J, “Adeno-Associated Virus Expression Systems for Gene Transfer,” Curr. Opin. Biotechno, October 1998; 9(5): 470-5; Samulski R J, Chang L S, Shenk T., “Helper-Free Stocks of Recombinant Adeno-Associated Viruses: Normal Integration Does Not Require Viral Gene Expression,” J. Virol. September 1989; 63(9):3822-8. Monahan P E, Samulski R J., “Adeno-Associated Virus Vectors for Gene Therapy: More Pros Than Cons?” Mol. Med. Today. November 2000; 6(11):433-40 (9, 10, 11), rAAV-Mediated gene delivery system is underway in clinical trials from Phase I to Phase III in several United States hospitals. See Cathomen T., “AAV Vectors for Gene Correction,” Curr. Opin. Mol. Ther. August 2004; 6(4):360-6.

### SUMMARY OF THE INVENTION

Systemic injection of 1012 AAV-shRNA vectors leads to reduction of HBV viral load at least 100 fold in hydrodynamic tranfection nude mice. Administration of AAV-shRNA vectors with 3TC displayed synergistic anti-HBV effects. Administration of AAV-shRNA vectors also inhibited HBV gene expression and improved liver functions in immunocompetent transgenic mice and reduced HBsAg- and HBx-induced liver cancer.

More specifically, the invention relates to a method for spontaneous delivery of two shRNAs targeting S and X region S both in vitro and in vivo to combat HBV replication and infection. The method involves administering an effective amount shRNAs to inhibit HBV replication in HepG2 cells. Those shRNAs are generated by human U6 promoter. Next, one, two or three shRNA expression cassettes are subcloned into an AAV plasmid and used to perform anti-HBV assays in HepG2 cells. All the constructs show potent anti-HBV activities. One construct with two shRNAs cassettes appears to show the best effect. Next, AAV-shRNA vectors are packaged with the plasmid containing two shRNA expression cassettes and a helper plasmid pDG. Also, AAV-shRNA vectors inhibit HBV replication in stable HBV reproducing cells HepAD38. Further, AAV-shRNA vectors inhibit HBV transcription and replication in HBV-producing nude mice. AAV-shRNA vectors exhibit synergistic anti-HBV effects with 3TC, and they inhibit HBsAg and HBx gene expression, improve liver functions, and reduce HBsAg- and HBx-induced liver cancer.

### BRIEF DESCRIPTION OF THE DRAWINGS

Other objects and features of the present invention will become apparent from the following detailed description of the preferred embodiments of the invention, considered in connection with the accompanying drawings in which:

FIG. 1 is the diagram of pAAV-shRNA constructs;

FIG. 2 shows that shRNAs generated by pAAV-shRNA constructs inhibits HBV replication in HepG2 cells in vitro:

FIG. 3 shows that AAV-EGFP vectors effectively transduce HBV-reproducing cells HepAD38;

FIG. 4 shows inhibition of HBV reproduction by administration of AAV-shRNA vectors (AAV-157i/1694i);

FIG. 5 shows marked reduction of HBsAg in the hepatocytes in the liver by administration of AAV-shRNA vectors in nude mice;

FIG. 6 shows marked plasma reduction of HBV viral load by administration of AAV-shRNA vectors in nude mice, and synergistic anti-HBV effects by administration of AAV-shRNA vectors (AAV-157i/1694i) with 3TC;

FIG. 7 shows reduction of mRNA level by administration of AAV-shRNA vectors in immunocompetent HBx-knock in mice;

FIG. 8 shows reduction of mRNA level by administration of AAV-shRNA vectors in immunocompetent HBsAg-knock in mice; and

FIG. 9 shows liver function improvement by administration of AAV-shRNA vectors in immunocompetent HBsAg- and HBx-knock in mice.

### DETAILED DESCRIPTION THE PREFERRED EMBODIMENTS

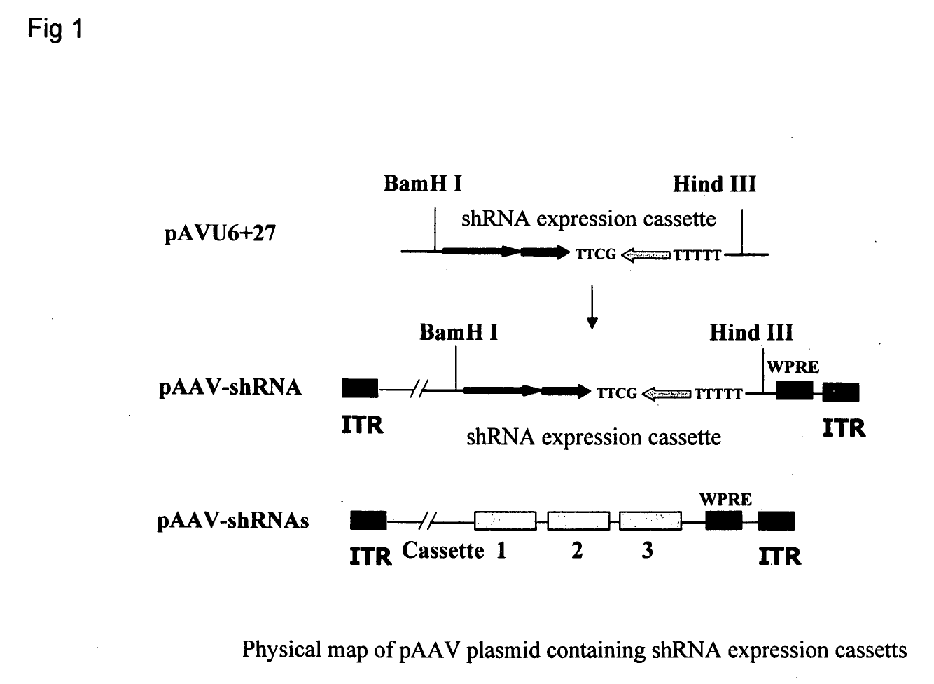

Referring to the drawings, FIG. 1 shows the pAAV-shRNA constructs of the present invention. The constructs expressing effective shRNAs targeting multiple sites of HBV genome are described. See Chen Y, Du D, Wu J, Chan C P, Tan Y, Kung H F, He M L, “Inhibition of Hepatitis B Virus Replication by Stably Expressed shRNA,” Biochem. Biophys. Res. Commun. November 14, 2003; 311 (2):398-404. The shRNA expression cassettes with U6 promoter are released with BamH I and Hind III digestion and ligated with linearized pAAV1 vector digested with the same enzymes. To generate a vector simultaneously expressing two shRNAs targeting different genes of HBV, DNA fragment with the second shRNA cassette (e.g. 1694i) released with BamH I and Hind III digestion are filled in with T4 DNA polymerase, and inserted into EcoR V site downstream the first shRNA cassette (e.g. 157i) of the multiple cloning sites (MCS) of pAAV1 with blunt-end ligation. The viral inverted terminal repeats (ITR) containing essential signals for viral replication and packaging are placed at each side of the expression cassette. Therefore, the recombinant virus genome only contains the expression cassette, WPRE elements, and viral ITR sequence, which is free of any other AAV viral genome. pAAV-shRNA plasmids (pAAV-157i, pAAV-736i, pAAV-1694i, pAAV-157i/1694i and pAAV-157i/736i/1694i) are generated.

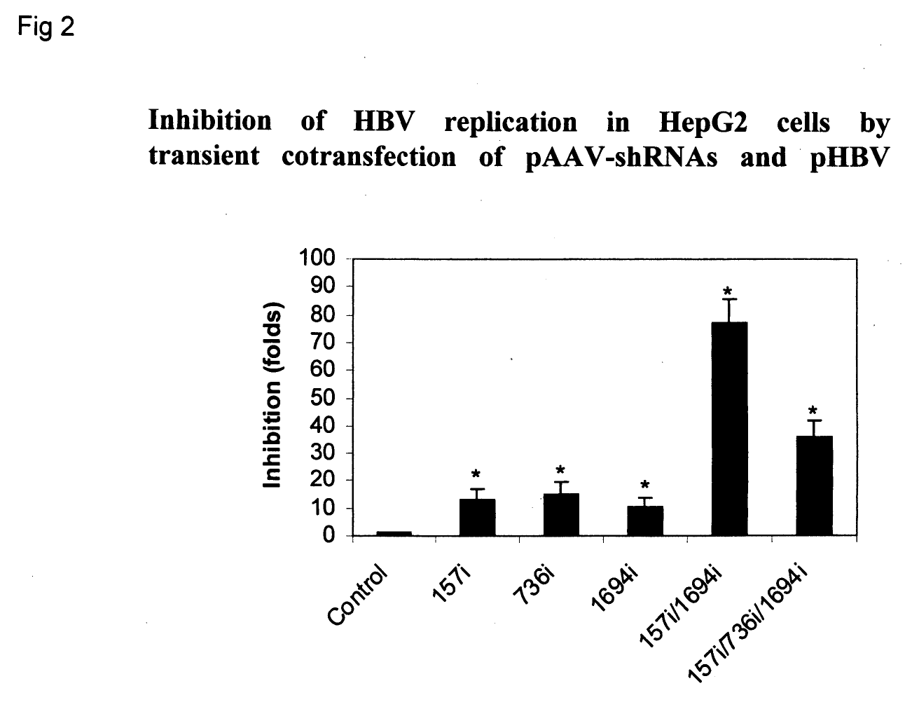

FIG. 2 shows shRNAs generated by pAAV-shRNA plasmids markedly inhibit HBV replication. We first screen most potent shRNA constructs by detecting their in vitro antiviral activities before we develop viral package system. HepG2 cells (ATCC, Manassas, Va.) are maintained in Dulbecco's modified Eagle's medium (DMEM), supplemented with 10% fetal bovine serum (FBS, Sigma, St. Louis, Mo.), 1% penicillin/streptomycin (PS), and 1% glutamine and incubated at 37° C. under 5% CO₂. We co-transfect HBV producing plasmid (pHBV), a luciferase expression plasmid (internal control for normalization of transfection efficiency) and an AAV plasmid containing either shRNA expression cassette(s) described above or U6 promoter (control) only into HepG2 cells using LipoFectamine 2000 (Invitragon, Calif.), according to manufacturer's instructions. Seventy-two hours post-transfection, we isolate intracellular HBV DNA in the viral particles and measure the copy numbers with quantitative PCR assay. Our results show that each individual shRNA inhibits viral replication at least 10 fold (n=4, P<0.01). The construct with 157i and 1694i cassettes showed over 50-fold inhibition, while the construct with three siRNA cassettes only displayed about 35 fold inhibition. Therefore, we choose dual siRNAs 157i/1694i to further develop AAV gene delivery system for gene therapy.

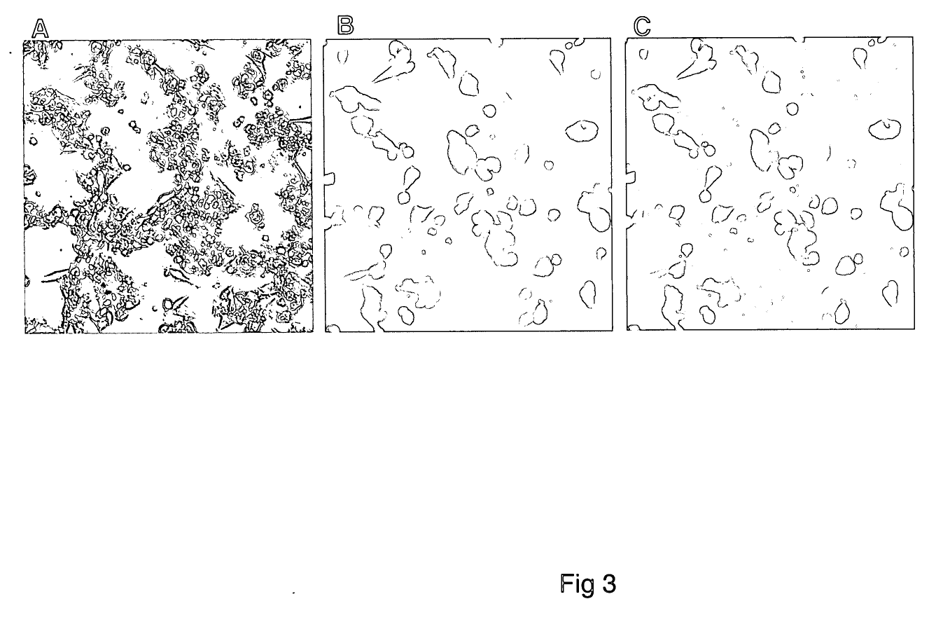

FIG. 3 shows the transduction efficiency of AAV vectors to HBV-reproducing HepAD38 cells using EGFP as an indicator. HEK 293 cells (ATCC, Manassas, Va.) are maintained in Dulbecco's modified Eagle's medium (DMEM), supplemented with 10% fetal bovine serum (FBS, Sigma, St. Louis, Mo.), 1% penicillin/streptomycin (PS), and 1% glutamine and incubated at 37° C. under 5% CO₂. Approximately 1.5×10⁶ of HEK 293 cells are seeded in 100-mm culture dishes and incubated at 37° C. under 5% CO₂ overnight. The cells reach about 80% confluence are co-transfected with 5 μg pAAV-shRNA or pAAV-EGFP (control) and 20 μg MV helper plasmid pDG using calcium phosphate co-precipitation method. Seventy two hours post-transfection, the cells are harvested and the recombinant AAV viruses are purified described by Wu et al. See Wu X, Dong X, Wu Z, Cao H, Niu D, Qu J, Wang H, Hou Y, “A Novel Method for Purification of Recombinant Adeno-Associated Virus Vectors on a Large Scale,” Chin. Sci. Bull. 2001; 46:485-489.

Briefly, 10% (v/v) chloroform is added to the cells and strongly vortexed for 30 min to release AAV virus from the cells. The supernatant is then collected after centrifugation at 12,000 rpm for 15 min. rAAV particles are precipitated with polyethylene glycol (PEG) 8,000. The pellets are then re-suspended in PBS buffer, and contaminated nucleotide acids are digested with 1 μg/ml Dnase I and RNase at 37° C. for 30 min. The AAV particles are further purified by chloroform and the viral genome copies are determined by quantitative real-time PCR (qPCR). qPCR is carried out with a set of primers and probe targeting WPRE region. The primers are 5′-CGG CTG TTG GGC ACT GA-3′ (forward) (SEQ. ID NO. 14) and 5′-CCG AAG GGA CGT AGC AGA AG-3′ (reverse) (SEQ. ID NO. 15). The probe was 5′-FAM-ACG TCC TTT CCA TGG CTG CTC GC-TMRA-3′ (SEQ. ID NO. 16).

HepAD38 cells are maintained in DMEM supplemented with 10% FBS, 1% PS, 1% glutamine, lOOpg/ml kanamycin, 400 μg/ml G418 and 0.3 μg/ml tetracycline. HepAD38 is a hepatocyte-derivated cell line with HBV reproduction. Such reproduction can be suppressed by addition of tetracycline in the culture medium and released by withdrawal from the culture medium. In order to facilitate the persistency of shRNA expression, we choose adeno-associated virus as our shRNA delivery vehicle, as it is believed to be one of most promising vectors for gene therapy. We package AAV viral vectors in HEK 293 cells and examine the transduction efficiency on hepatocyte-derivated HepAD38 cells using AAV-EGFP with different MOI. HepAD38 cells were infected with rMV-EGFP and FIG. 3 shows images of the cells 48 hours postinfection by phase-contrast microscopy (A), fluorescence microscopy (B), and by overlay of images A and B. We observe that 10⁵ MOIs of AAV-EGFP display the strongest fluorescence 48 hours after transduction (FIG. 3). About 90% of cells are transduced by AAV-EGFP vectors.

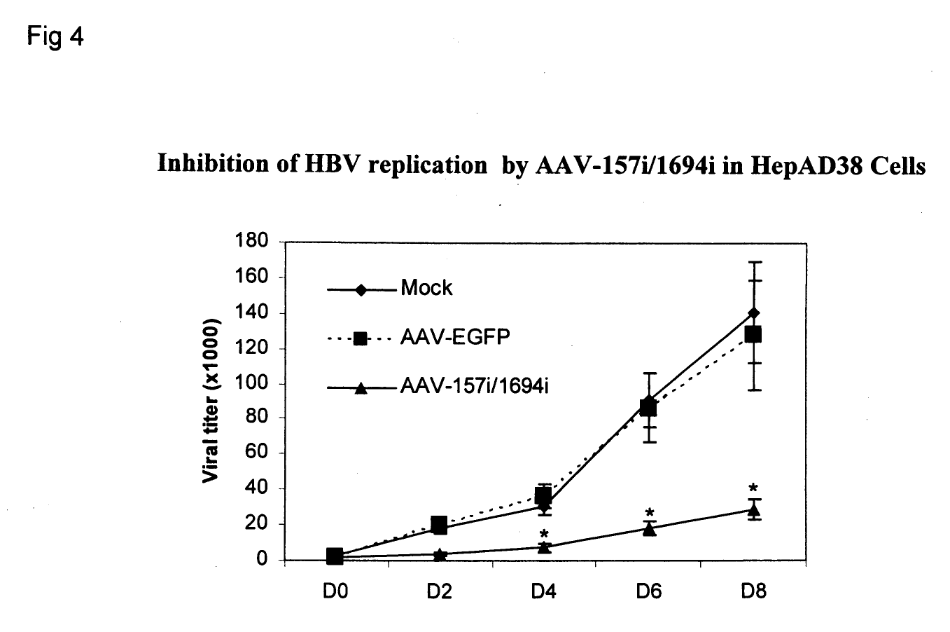

FIG. 4 shows inhibition of HBV replication by AAV-shRNA (AAV-157i/1694i) vectors in vitro in HepAD38 cells. We determined the anti-HBV activities of AAV vector carrying shRNA 157i/1694i expression cassettes in vitro. We infect HepAD38 cells with AAV-157i/1694i at 10⁵ multiplicity of infection (MOI, viral genome). After AAV transduction, the extracellular HBV DNA is isolated for viral quantification at different time points. Before the transduction, HepAD38 is cultured with tetracycline to suppress the HBV production. The viral titers in the medium is measured with real-time PCR assay and recorded as day 0. After transduction, the tetracycline is removed and HBV DNA is collected from culture medium. We find that nearly 80% of the extracellular HBV DNA is reduced compared with the mock and AAV-EGFP control 2 days post-transduction, and the same level inhibition is maintained at least 8 days (FIG. 4).

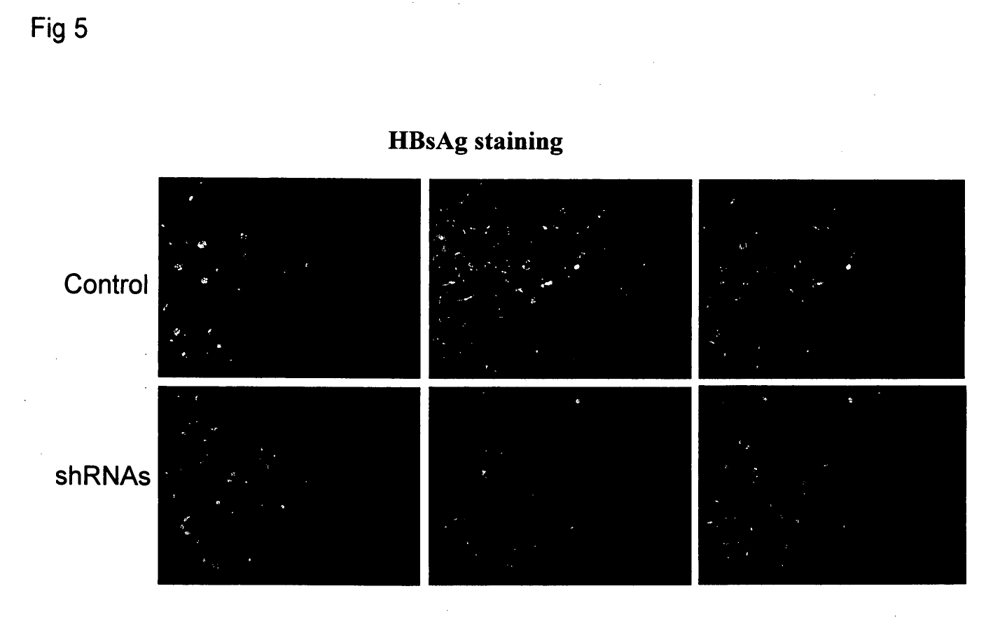

FIG. 5 shows reduction of HBsAg level in hepatocytes in vivo. The hydrodynamic transfection HBV model was used in this study (6 mice for each group). The plasmid pHBV (Adr subtype), which is used to generate HBV viruses in mice liver, is prepared by QIAGEN plasmid maxi kit according to the manufacturer's protocol. PBS, 10¹² of viral particles (viral genome, vg) AAV-EGFP, or AAV-shRNA-157i/1694i are administered via the tail vein of 4-8 week old female nude mice. One week later 40 μg plasmid are injected into the tail vein in a volume of PBS about 10% of its body weight (1 ml of 10 g-mice) within 5-7 seconds. The mice are sacrificed at 1, 2 and 3 weeks to detect HBsAg level by immunohistochemistry assays. Liver tissue is fixed with 4% paraformaldehyde overnight at 4° C. To detect the transgene expression, EGFP is directly observed on the sections under a fluorescence microscope. To examine the expression level of HBsAg and HBV, the sections are rinsed with PBS containing 0.1% Triton TX-100 three times. The sections are blocked with normal bovine serum, and incubated with a primary monoclonal antibody against HBsAg and HBV. After hybridization with a FITC-conjugated secondary antibody, the image is recorded under a fluorescence microscope. The left panels show the cell numbers stained with DAPI, the middle panels show the HBsAg level, and we observe that HBsAg is highly expressed in hepatocytes in the control group (upper panel), whereas HBsAg is reduced to almost undetectable level in the test group (lower panel). Right panels show the overlay signals.

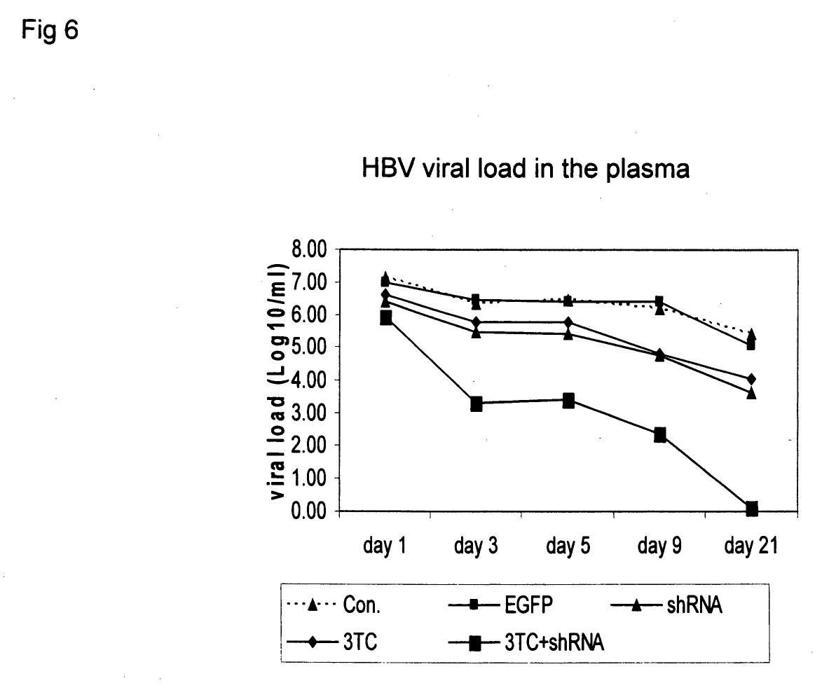

FIG. 6 shows potent reduction of viral load in the plasma by administration of AAV-shRNA vector in hydrodynamic transfection HBV mice. PBS, 10¹² of viral particles (viral genome, vg) AAV-EGFP, or AAV-shRNA-157i/1694i are administered via tail vein of 4-8 weeks old female nude mice, and one week later 40 μg of plasmid are injected into the tail vein in a volume of PBS about 10% of its body weight (1 ml of log-mice) within 5-7 seconds. Either AAV-EGFP or PBS is used as a control. After 1 day of HBV plasmid injection, blood is collected from mice tail vein and the level of the surface antigen (S protein) of HBV viral particles is measured by ELISA with an ELISA kit (Murex HBsAg Version 3 Kit, Abbott Murex, UK) according to the manufacturer's protocol. As shown in Table 1, the hydrodynamic transfection mice which are post-injection of AAV-shRNA-157i/1694i, HBsAg level remains at a very low level after HBV plasmid injection at day 1, and reduces to undetectable level from day 3 to day 21 in most mice. While in the control groups, plasma HBsAg level is maintained very high from day 1 to day 9. At day 21, the plasma HBsAg is maintained relative high levels in most mice. HBV DNA is a golden marker of HBV level because it reflects the viral load in the plasma. We therefore also quantify plasma HBV DNA. As shown in FIG. 6, the viral load drops about 10 fold at day 1 and about 100 fold at day 21 in the plasma.

We also examine the synergistic antiviral effects of shRNA and 3TC. To examine whether shRNA gene therapy displays synergistic antiviral effects with chemotherapy, we inject 3TC into mice (s.c.) at 1 mg/1 kg body weight/day for 3 weeks after hydrodynamic transfection of pHBV plasmid. Then we monitor HBsAg and HBV DNA level in the plasma. Compared with the groups treated with either AAV-EGFP, 3TC alone or untreated group, we find that HBsAg level is significantly reduced in the plasma in the group treated with AAV-157i/1694i and lamivudine (Table 1, P<0.01). After we quantify the HBV DNA, we find over hundreds to thousand fold inhibition compared with AAV-EGFP or PBS control, or over 10 to 100 fold additional inhibition compared to AAV-157i/1694i and 3TC treatment (FIG. 6).

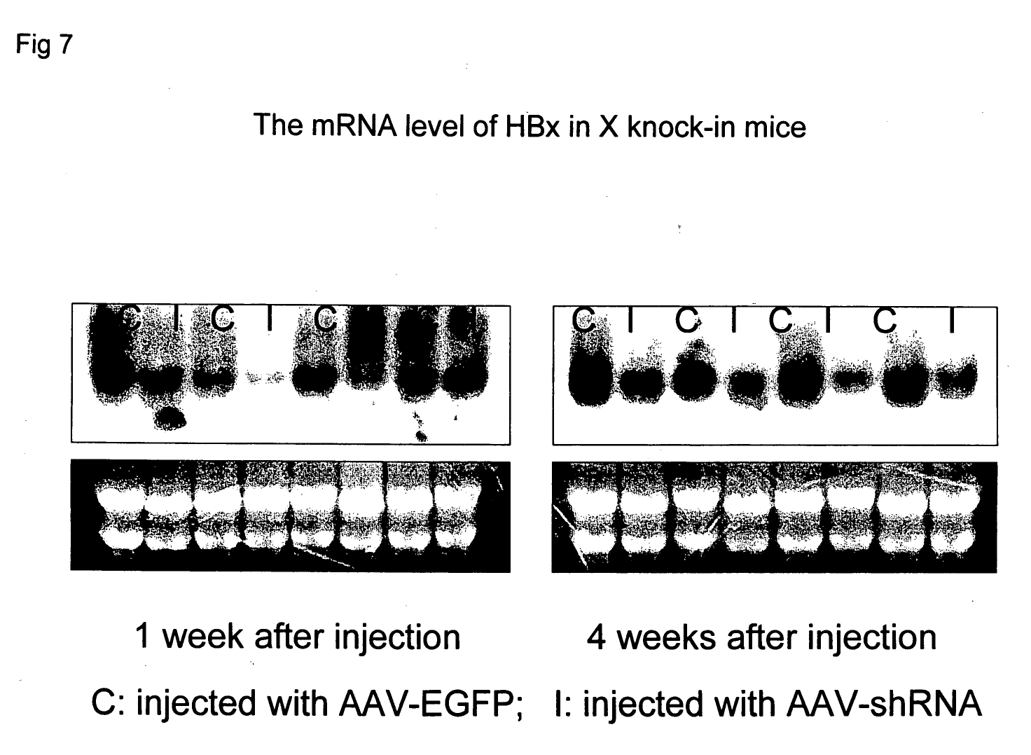

FIG. 7 shows inhibition of HBx gene transcription after administration of AAV-shRNA vectors in HBx knock in mice. To investigate whether long term expressed shRNA delivered with AAV vectors could inhibit S or X induced liver cancer, we use transgenic mice models. In the mice, HBV X gene is knocked in the p21 locus leading to constant expression of HBx protein in mice liver and disrupting p21 expression. These mice would develop HCC about one year after birth. See Wang Y, Cui F, Lv Y, Li C, Xu X, Deng C, Wang D, Sun Y, Hu G, Lang Z, Huang C, Yang X, “HBsAg and HBx Knocked Into the p21 Locus Causes Hepatocellular Carcinoma in Mice,” Hepatology, February 2004; 39(2):318-24. We inject either AAV-EGFP or AAV-157i/1694i vectors (10¹² v.g./mice) into the tail vein of 4- to 6-week old transgenic mice. These mice are immunocompetent. One week or four weeks post-injection, the mice are sacrificed to collect liver for X expression assay and to collect plasma for a liver function assay. Northern blot experiments shows that mRNA level of X gene is obviously reduced in three of four mice in the X gene knock-in mice group treated with AAV-157i/1694i and sacrificed after one week injection, and all four mice in the group treated with AAV-157i/1694i sacrificed four weeks post-injection.

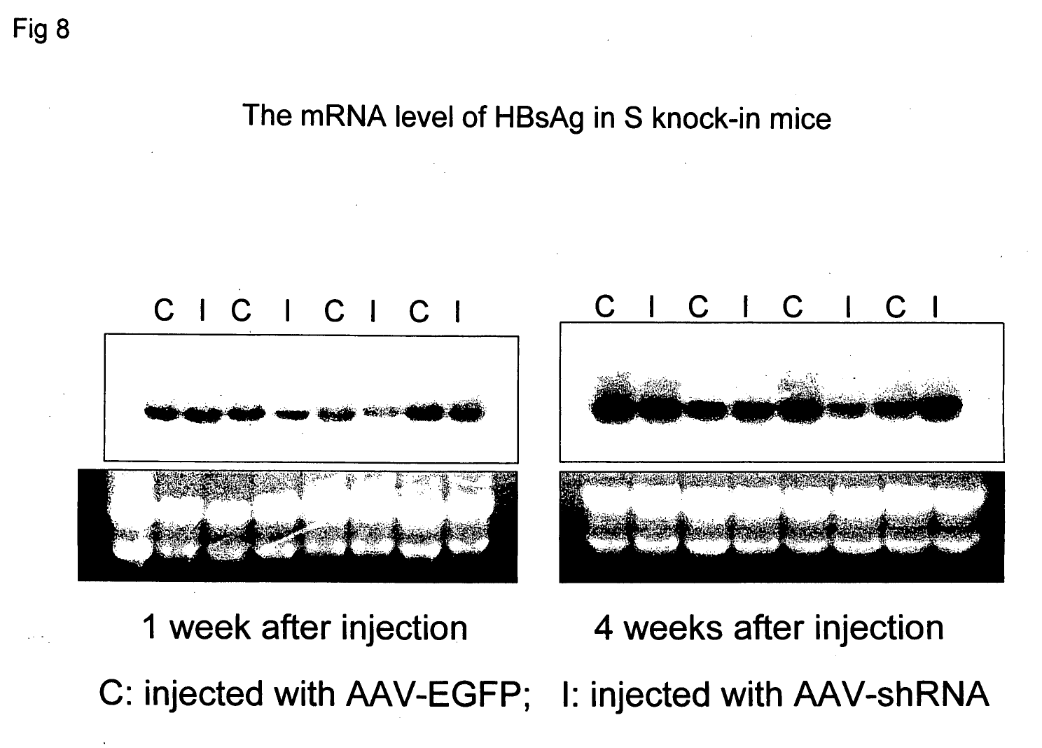

FIG. 8 shows inhibition of HBsAg gene transcription after administration of AAV-shRNA vectors. We use transgenic mice models. In the mice, HBV S gene is knocked in p21 locus leading to constant expression of HBsAg protein in mice liver and disrupts p21 expression. Those mice would develop HCC about one year after birth. See Wang Y, Cui F, Lv Y, Li C, Xu X, Deng C, Wang D, Sun Y, Hu G, Lang Z, Huang C, Yang X, “HBsAg and HBx Knocked Into the p21 Locus Causes Hepatocellular Carcinoma in Mice,” Hepatology, February 2004;39(2):318-24. We inject either AAV-EGFP or AAV-157i/1694i vectors (1012 v.g./mice) into the tail vein of 4- to 6-week old transgenic mice. These mice are immunocompetent. One week or four weeks post-injection, the mice are sacrificed to collect their liver for S expression assay and to collect plasma for liver function assay. Northern blot experiments shows that mRNA level of S gene is obviously reduced in three of four mice in the S gene knock-in mice group treated with AAV-157i/1694i and sacrificed after one week injection, and all four mice in the group treated with AAV-157i/1694i sacrificed four weeks post-injection.

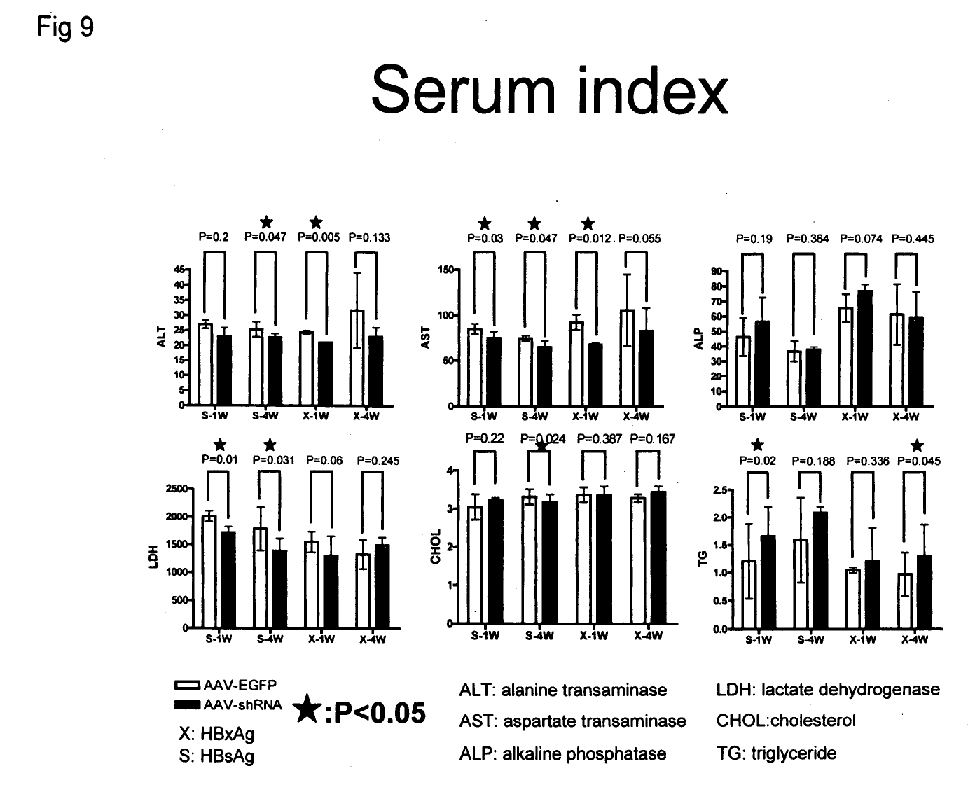

FIG. 9 shows the improvement of liver functions after administration of AAV-shRNA vector into S or X knock-in mice described in the description of FIG. 7 and FIG. 8. The levels of sera alanine transaminase (ALT), aspartate transaminase (AST), alkaline phosphatase (ALP), lactate dehydrogenase (LDH), cholesterol (CHOL), and triglyceride (TG) reflect liver functions. Liver function assays also show that stably expressed shRNA delivery by AAV vectors improve liver functions. For HBsAg knock-in mice, plasma alanine transaminase (ALT), aspartate transaminase (AST), alkaline phosphatase (ALP), lactate dehydrogenase (LDH), and cholesterol (CHOL) and triglyceride (TG) are significantly reduced four weeks post-AAV administration (Table 2 and FIG. 9, P<0.05), compared with control groups. One week post-injection, S knock-in mice showed significant decrease of LDH and AST activity. The results also show that a significant decrease of ALT and CHOL level at 4 weeks post-injection in HBsAg transgenic mice; and a significant increase of TG level at 1 week post-injection. For X knock in mice, ALT and AST levels are significantly reduced at 1 week, and TG level is significant increased at four weeks post administration of AAV-shRNA vectors. The detailed data is shown in TABLE 2 shows parameters of liver functions after administration of AAV-shRNA vectors in immunocompetent HBsAg-in and HBx-knock in mice.

We also observe HCC formation after AAV administration. For S knock-in mice, four mice developed HCC in the control group and only one mouse developed HCC in AAV-157i/1694i group (n=6). For X knock-in mice, four mice developed HCC in the control group and only two mice develop HCC (n=6).

The AAV-shRNA vectors to be delivered, will be administered systemically, most typically by intravenous, intraperitoneal or intratumor injection, in an amount effective for delivery of shRNAs targeting a specific gene. AAV-shRNA vectors are also administered orally, or via respiratory tract.

Experimental Section

Although described primarily with reference to delivery of therapeutics, it should be recognized by those skilled in the art that the same delivery system can be used for laboratory use to transduce cells. For example, AAV-shRNA vectors can be used to probe a new gene functions by silencing a specific gene (loss of function) or to elucidate gene signaling pathways. The present invention will be further understood by reference to the following non-limiting examples.

### EXAMPLE 1

To probe new gene functions: A new gene is suspected to be an oncogene, which may be involved in hepatocarcinogenesis. One can first design shRNAs to target this gene. Next, oligos coding for these shRNAs are chemically synthesized. Next, oligos are annealed and inserted into multiple cloning sites of pAVU6+27 plasmid to generate shRNA expression cassettes. Next, the shRNA expression cassettes are subcloned into pAAV plasmid and packaged into AAV-shRNA vectors. After that, HCC cell lines (such as HepG2, Huh7, Hep3B etc) are infected with AAV-shRNA vector with different MOls. Target gene expression level is checked by RT-PCR or western blot, as is cell proliferation, and apoptosis.

### EXAMPLE 2

Gene therapy for chronic infectious diseases, such as HCV. First, one can design shRNAs to target HCV RNA. Next, AAV-shRNA vectors are generated. Next, HCV reproducing cells are infected with AAV-shRNAs, and anti-HCV efficacy is measured with ELISA assay and real time RT-PCR assay. Next, animals or patients are given a certain amount of MV-shRNA vectors systemically or via portal vein. Next, viral load and/or liver functions are monitored.

### EXAMPLE 3

Gene therapy for cancers, such as liver cancer. If gene A is a specific oncogene contributing to hepatocarcinogenesis, and is essential to maintain HCC growth, then specific shRNAs targeting gene A will be designed and AAV-shRNA vectors will be generated. AAV-shRNA vectors will be injected intravenously or intratumorly. The vectors can be injected weekly or monthly depending on the effects checked under CT (patents).

Additional Examples

Constructs

pAVU6+27, which contains human U6 promoter and the first 27-bp of U6 RNA coding sequence, has been described by Paul et al., 2002, Nat Biotechnol 20:505-508. A series of shRNA expression vectors was generated by inserting annealed oligos containing sense-TTCG-antisense sequence into pAVU6+27 vector between Sal I and Xba I sites.

The oligo sequences coding for the sense strand of shRNA were:

| | |
|---|---|
| 87i, | |
| 5′-GACTACTGCCTCACCCATA-3′; | (SEQ ID NO:1) |
| 157i, | |
| 5′-CATGGAGAGCACAACATCA-3′; | (SEQ ID NO:2) |
| 451i, | |
| 5′-GACTACCAAGGTATGTTGC-3′; | (SEQ ID NO:3) |
| 660i, | |
| 5′-CGTTTCGCCTGGCTCAGTT-3′; | (SEQ ID NO:4) |
| 736i, | |
| 5′-GTTATATGGATGATGTGGT-3′; | (SEQ ID NO:5) |
| 1593i, | |
| 5′-TTCACCTCTGCACGTCGCA-3′ | (SEQ ID NO:6) |
| (target DR2); | |
| 1694i, | |
| 5′-GACCTTGAGGCATACTTCA-3′; | (SEQ ID NO:7) |
| 1826i, | |
| 5′-TTCACCTCTGCCTAATCAT-3′ | (SEQ ID NO:8) |
| (target DR1); | |
| 2310, | |
| 5′-GTTGATAAGATAGGGGCAT-3′; | (SEQ ID NO:9) |
| and | |
| 2979i, | |
| 5′-ACTTCAACCCCAACAAGG-3′. | (SEQ ID NO:10) |

Cell Culture, Transfection, and Reporter Gene Assays

HepG2 cells were grown in DMEM with 10% fetal bovine serum in 10-cm dishes. Transfections were carried out using Lipofectamine 2000 reagent (Invitrogen, MD) as described in the manufacturer's instructions. The transfected cells were selected with 500 μg/ml of G418 for three weeks with medium changes every three days. The cells for the stable expression of shRNA were used for HBV replication assay.

To detect the effects of stably-expressed shRNA on HBV RNA degradation and replication, HepG2 cells were cotransfected with 100 ng of luciferase expression plasmid pJMD1948 (He et al., 1999, Proc Natl Acad Sci USA 96:10212-10217) and 900 ng of pHBV (Fu et al., 1998, Biochem Pharmacol 55:1567-1572; Fu et al., 1997, Chin J. Virol., 13:215-223) in each well of 12-well plates. Luciferase activities were determined after 72 hrs using a luciferase detection kit (Promega, WI). The HBV titers were normalized by luciferase activities. To test the synergistic effects of shRNA and lamivudine (3TC), the shRNA stable-expression cells cotransfected with pHBV and pJDM1948 were culture in 3TC containing medium (0.5 μM) and harvested after 6-day incubation with fresh medium change every two days.

Quantitative PCR Analysis

Real-time PCR was performed to quantify HBV viral genomic DNA or mRNA using an HBV diagnostic kit (PG Biotech. Ltd., Shenzheng, China) described previously. He et al., 2002, Biochem Biophys Res Commun 295:1102-1107. For measurement of viral genomic DNA, HepG2 cells were harvested 72 hrs post-transfection and lysed in 200 μl of lysis buffer (PBS with 1% NP-40 and cocktail protein inhibitors). The luciferase activities were determined using 25 μl of cell lysates. The remaining cell lysates were treated with DNase I (final conc. 1 mg/ml) at 37° C. for 60 min to remove the transfected plasmid DNA before the isolation of HBV genomic DNA from core particles. To quantify the mRNA of HBV, the total mRNA was isolated using Trizol reagent (Invitrogen, MD) and reverse-transcribed (RT) to cDNA using oligo dT primimg. Quantitative RT-PCR experiments were carried out and the values were normalized with luciferase mRNA (internal control). To quantify luciferase mRNA (internal control), primer 5′-GCGACCAACGCCTTAGATTG CAA-3′ (Luc_F) (SEQ ID NO:11), 5′-GCGGTCAACG ATGAAGAAGTG-3′ (Luc_R) (SEQ ID NO:12) and probe 5′-FAM-ATGGATGGCTACATTCTGGA GACATAG-TAMRA-3′ (SEQ ID NO:13) were used in the real-time PCR reactions.

### REFERENCES

The following references are incorporated herein by reference:
1. Lau G K. Hepatitis B infection in China. Clin. Liver Dis. May; 5(2):361-379.
2. Chen Y, Du D, Wu J, Chan C P, Tan Y, Kung H F He M L. Inhibition of hepatitis B virus replication by stably expressed shRNA. Biochem. Biophys. Res. Commun. Nov. 14, 2003; 311(2):398-404.
3. Ying C, De Clercq E, Neyts J. Selective inhibition of hepatitis B virus replication by RNA interference. Biochem Biophys Res Commun. Sep. 19, 2003;309(2):482-4.
4. McCaffrey A P, Nakai H, Pandey K, Huang Z, Salazar F H, Xu H, Wieland S F, Marion P L, Kay M A. Inhibition of hepatitis B virus in mice by RNA interference. Nat Biotechnol. June 2003;21(6):639-44.
5. Zhang X N, Xiong W, Wang J D, Hu Y W, Xiang L, Yuan Z H. siRNA-mediated inhibition of HBV replication and expression. World J Gastroenterol. Oct. 15, 2004; 10(20):2967-71.
6. Ni Q, Chen Z, Yao H P, Yang Z G, Liu K Z, Wu L L. Inhibition of human La protein by RNA interference downregulates hepatitis B virus mRNA in 2.2.15 cells. World J Gastroenterol. Jun. 15, 2004; 10(14):2050-4.
7. Liu J, Guo Y, Xue C F, Li Y H, Huang Y X, Ding J, Gong W D, Zhao Y. Effect of vector-expressed siRNA on HBV replication in hepatoblastoma cells. World J Gastroenterol. Jul. 1, 2004; 10(13):1898-901.
8. Morrissey D V, Lockridge J A, Shaw L, Blanchard K, Jensen K, Breen W, Hartsough K, Machemer L, Radka S, Jadhav V, Vaish N, Zinnen S, Vargeese C, Bowman K, Shaffer C S, Jeffs L B, Judge A, MacLachlan I, Polisky B. Potent and persistent in vivo anti-HBV activity of chemically modified siRNAs. Nat Biotechnol. August 2005; 23(8):1002-7.
9. Rabinowitz J E, Samulski J. Adeno-associated virus expression systems for gene transfer. Curr Opin Biotechnol. October 1998; 9(5):470-5.
10. Samulski R J, Chang L S, Shenk T. Helper-free stocks of recombinant adeno-associated viruses: normal integration does not require viral gene expression. J Virol. September 1989; 63(9):3822-8.
11. Monahan P E, Samulski R J. Adeno-associated virus vectors for gene therapy: more pros than cons? Mol Med Today. November 2000; 6(11):433-40.
12. Cathomen T. AAV vectors for gene correction. Curr Opin Mol Ther. August 2004; 6(4):360-6.
13. Wu X, Dong X, Wu Z, Cao H, Niu D, Qu J, Wang H, Hou Y. A novel method for purification of recombinant Adeno-associated virus vectors on a large scale. Chin Sci Bulle. 2001; 46:485-9.
14. Yang P L, Althage A, Chung J, Chisari F V. Hydrodynamic injection of viral DNA: a mouse model of acute hepatitis B virus infection. Proc Natl Acad Sci USA. Oct. 15, 2002; 99(21):13825-30. Epub Oct. 8, 2002.
15. Moore M D, McGarvey M J, Russell R A, Cullen B R, McClure M O. Stable inhibition of hepatitis B virus proteins by small interfering RNA expressed from viral vectors. J Gene Med. July 2005; 7(7):918-25.
16. Cheng T L, Chang W W, Su I J, Lai M D, Huang W, Lei H Y, Chang W T. Therapeutic inhibition of hepatitis B virus surface antigen expression by RNA interference. Biochem Biophys Res Commun. Oct. 28, 2005; 336(3):820-30.
17. Lee M T, Coburn G A, McClure M O, Cullen B R. Inhibition of human immunodeficiency virus type 1 replication in primary macrophages by using Tat- or CCR5-specific small interfering RNAs expressed from a lentivirus vector. J Virol. November 2003; 77(22):11964-72.
18. Spagnou S, Miller A D, Keller M. Lipidic carriers of siRNA: differences in the formulation, cellular uptake, and delivery with plasmid DNA. Biochemistry. Oct. 26, 2004; 43(42):13348-56.
19. Anderson J, Akkina R. HIV-1 resistance conferred by siRNA cosuppression of CXCR4 and CCR5 coreceptors by a bispecific lentiviral vector. AIDS Res Ther. Jan. 13, 2005; 2(1):1.
20. Wang Y, Cui F, Lv Y, Li C, Xu X, Deng C, Wang D, Sun Y, Hu G, Lang Z, Huang C, Yang X. HBsAg and HBx knocked into the p21 locus causes hepatocellular carcinoma in mice. Hepatology. February 2004;39(2):318-24.
21. Wu H L, Huang L R, Huang C C, Lai H L, Liu C J, Huang Y T, Hsu Y W, Lu C Y, Chen D S, Chen P J. RNA interference-mediated control of hepatitis B virus and emergence of resistant mutant. Gastroenterology. March 2005; 128 (3):708-16.

We claim:

1. An isolated nucleic acid molecule which hybridizes under stringent conditions to an isolated nucleic acid molecule comprising the nucleotide sequence of SEQ ID NO: 1, or 10, or a complement thereof.

2. A method of treatment for a disease related to HBV in a subject in need thereof comprising administering to the subject a nucleic acid molecule comprising the nucleotide sequence of SEQ IS NO: 1, or 10, or a complement thereof.

3. The method of claim 2, further comprising administering to the subject lamivudine and/or interferon alpha.

4. A method for treating a disease caused by HBV in a subject in need thereof, comprising administering to the subject a vector comprising an isolated nucleic acid molecule comprising the nucleic acid sequence SEQ ID No: 1, 2, 3, 4, 7 or 10.

5. The method of claim 4, wherein the nucleic acid molecule is operatively linked to human U6 promoter.

6. The method of claim 4, wherein the nucleic acid molecule comprises a sense-TTCG-antisense sequence of the nucleotide sequence.

7. A method of treatment for a disease related to HBV in a subject in need thereof, comprising administering to the subject the nucleic acid molecule of claim 1, wherein the nucleic acid molecule comprises a sense-TTCG-antisense sequence of the nucleotide sequence.

8. The method of claim 7, wherein the nucleic acid molecule is a mRNA.

9. A method of inhibiting expression of a target gene of HBV in a host cell, comprising administering to the host cell the nucleic acid molecule of claim 1, wherein expression of the target gene is inhibited by 90% or more compared to the expression of the target gene before administering the nucleic acid molecule to the host cell.

10. A method for suppressing or inhibiting HBV replication, in liver cells infected therewith, comprising administering an effective amount of an AAV-shRNA vector in a pharmaceutically acceptable vehicle.

11. A method of inhibiting or suppressing HBV gene expression in a subject animal infected with HBV, comprising administering an amount of an AAV-shRNA vector effective to inhibit or suppress HBV gene expression in a pharmaceutically effective vehicle.

12. A method according to claim 11, wherein the subject animal is a mammal.

13. A method according to claim 12, wherein the mammal is a mouse.

14. A method in accordance with claim 12, wherein the mammal is a human.

15. A method according to claim 12, wherein 3TC or lamivudine is administered in addition to or together with the rAAV-shRNA vector.

16. A method according to claim 12, wherein the AAV-shRNA vector is rAAV-shRNA-157i/1694i.

17. A method for improving liver function in a subject mammal, comprising administering to the mammal an amount of an AAV-shRNA vector effective to improve liver function in the mammal.

18. A method for inhibiting HBsAg and HBx gene expression in HBV infected liver cells, comprising administering an effective amount of an AAV-shRNA to the HBV infected cells.


SEQUENCE LISTING (16 sequences)

SEQ ID NO: 1 - DNA, 19 bp, Artificial Sequence
  Synthetic oligonucleotide
gactactgcctcacccata

SEQ ID NO: 2 - DNA, 19 bp, artificial sequence
  synthetic oligonucleotide
catggagagcacaacatca

SEQ ID NO: 3 - DNA, 19 bp, artificial sequence
  Synthetic oligonucleotide
gactaccaaggtatgttgc

SEQ ID NO: 4 - DNA, 19 bp, artificial sequence
  synthetic oligonucleotide
cgtttcgcctggctcagtt

SEQ ID NO: 5 - DNA, 19 bp, artificial sequence
  synthetic oligonucleotide
gttatatggatgatgtggt

SEQ ID NO: 6 - DNA, 19 bp, artificial sequence
  synthetic oligonucleotide
ttcacctctgcacgtcgca

SEQ ID NO: 7 - DNA, 19 bp, artificial sequence
  synthetic oligonucleotide
gaccttgaggcatacttca

SEQ ID NO: 8 - DNA, 19 bp, artificial sequence
  synthetic oligonucleotide
ttcacctctgcctaatcat

SEQ ID NO: 9 - DNA, 19 bp, artificial sequence
  synthetic oligonucleotide
gttgataagataggggcat

SEQ ID NO: 10 - DNA, 18 bp, artificial sequence
  synthetic oligonucleotide
acttcaaccccaacaagg

SEQ ID NO: 11 - DNA, 23 bp, artificial sequence
  synthetic oligonucleotide primer
gcgaccaacgccttagattgcaa

SEQ ID NO: 12 - DNA, 21 bp, artificial sequence
  synthetic oligonucleotide primer
gcggtcaacgatgaagaagtg

SEQ ID NO: 13 - DNA, 27 bp, artificial sequence
  synthetic oligonucleotide probe
atggatggctacattctggagacatag

SEQ ID NO: 14 - DNA, 17 bp, artificial sequence
  synthetic oligonucleotice forward primer
cggctgttgggcactga

SEQ ID NO: 15 - DNA, 17 bp, artificial sequence
  synthetic oligonucleotide reverse primer
aagggacgtagcagaag

SEQ ID NO: 16 - DNA, 23 bp, artificial sequence
  synthetic oligonucleotide probe
acgtcctttccatggctgctcgc